(19) United States
(12) Patent Application Publication
(10) Pub. No.: US 2020/0176115 A1
(43) Pub. Date: Jun. 4, 2020

(54) AN APPLICATION FOR EARLY PREDICTION OF PENDING SEPTIC SHOCK

(71) Applicant: THE JOHNS HOPKINS UNIVERSITY, Baltimore, MD (US)

(72) Inventors: Raimond L. Winslow, Baltimore, MD (US); Sridevi V. Sarma, McLean, VA (US); Ran Liu, Farmington, CT (US)

(21) Appl. No.: 16/636,396

(22) PCT Filed: Aug. 8, 2018
(86) PCT No.: PCT/US2018/045317

§ 371 Date: Feb. 4, 2020

Related U.S. Application Data
(60) Provisional application No. 62/541,238, filed on Aug. 4, 2017.

(51) Int. Cl. G16H 50/20; G16H 50/30; G16H 10/60

(52) U.S. Cl. CPC G16H 50/20; G16H 10/60; G16H 50/30

(57) ABSTRACT

The present invention is directed to a system and method for using physiological time-series (PTS) data sampled continuously from patients in the ICU. An algorithm according to an embodiment of the present invention applies statistical modeling and machine learning methods to implement an early warning policy for predicting those patients likely to transition from non-sepsis, early sepsis or sepsis into septic shock. Results demonstrate that the system and method of the present invention can provide higher sensitivity and specificity in this task than any other method reported to date. It provides an advanced early warning of this pending transition with median value 12.5 hours, giving ample opportunity for physicians to intervene to prevent the patient from developing septic shock.

### CROSS REFERENCE TO RELATED APPLICATION

This application claims the benefit of U.S. Provisional Patent Application No. 62/541,238 filed on Aug. 4, 2017, which is incorporated by reference, herein, in its entirety.

### FIELD OF THE INVENTION

The present invention relates generally to risk assessment. More particularly, the present invention relates to an application for early prediction of pending septic shock.

### BACKGROUND OF THE INVENTION

Sepsis is a life-threatening organ dysfunction caused by a dysregulated host response to infection. Septic shock is a subset of sepsis with profound circulatory, cellular, and metabolic abnormalities associated with a greater risk of mortality than sepsis alone. Sepsis and septic shock are the leading causes of hospital mortality, accounting for an estimated 37-56% of all inpatient deaths. Septic shock is particularly lethal, with mortality estimated as high as 45%. Timely treatment of septic shock is crucial in improving patient outcome. Patients with septic shock treated within the first hour of diagnosis had a survival rate of 80%, but for every hour that septic shock went untreated, mortality increased by ˜8%. This same study found that in many cases, there was a substantial delay between diagnosis and treatment, with average time to treatment in sepsis and septic shock being 6 hours.

Timely administration of antibiotics for septic patients has been shown to be life-saving. Moreover, the Surviving Sepsis Campaign recommends treatment protocols, known as sepsis bundles that are to be executed within specific time windows to treat patients with sepsis and septic shock. Several studies have demonstrated that when sepsis bundles are implemented as soon as possible following diagnosis, mortality of septic shock is reduced substantially.

Hospital patients, particularly those in critical care units, are heavily instrumented to monitor their physiological function. Physiological time-series (PTS) data, generated by continuous sampling of these sensor signals at both high (per-msec)) and low (per-sec) frequencies, are a rich source of moment-to-moment information that will provide the earliest possible indicators of a change in patient physiological state. Barriers to developing real-time early-warning risk scores based on these data are the lack of: (1) automated, scalable tools for reliably capturing patient PTS data linked with corresponding clinical data; and (2) lack of validated approaches for analyzing the complex and dynamic relationships between a patient's physiologic signals and clinical parameters to accurately predict risk.

Accordingly, there is a need in the art for an automated system that could detect and provide advanced notice of patient deterioration into septic shock to reduce time to treatment, and thus improve patient outcomes.

### SUMMARY OF THE INVENTION

The foregoing needs are met, to a great extent, by the present invention which provides a method for predicting septic shock in a patient including acquiring data for the patient, wherein the data comprises physiological time-series (PTS) data and electronic health record (EHR) data. The method includes determining a risk score for the patient at a predetermined time interval using a generalized linear model (GLM). The method also includes treating the risk score as the observable output of a hidden Markov model (HMM), using the HMM to estimate a transition probability that a patient has transitioned from a clinical state of sepsis to a pre-shock state. The transition probability is compared to a fixed threshold. The method includes classifying the patient's condition as septic shock if the patient reaches the fixed threshold, wherein the time at which the patient reaches the fixed threshold is defined as to and triggering a healthcare response if the patient reaches t_{d}.

In accordance with an aspect of the present invention, the PTS data includes heart rate, systolic blood pressure, partial pressure of oxygen in arterial blood, respiratory rate, Glasgow Coma Score, lactate level, blood urea nitrogen, white blood cell count, and respiratory, coagulatory and cardiovascular SOFA scores. The generalized linear model is defined as

$$\hat{P} ( t ) = \frac{{e}^{{β}_{0} + {\underline{β}}^{T} \underline{x} ( t )}}{1 + {e}^{{β}_{0} + {\underline{β}}^{T} \underline{x} ( t )}}$$

and the HMM is defined as π(t)=P(y(t)=1|x(t), x(t−1), . . . , x(1)), where π(t) is the time-evolving transition probability. The PTS data is acquired at least every minute, and the risk score is calculated at least every minute. The PTS data is being updated continuously. The risk score and transition probability are updated whenever a new clinical measurement becomes available in the PTS data or the EHR data. The threshold on transition probability is chosen to correspond to a point on a receiver operating curve (ROC) that is closest to a true positive rate (TPR)=1 and false positive rate (FPR)=0. Alternately, the transition probability is chosen based on a detection rule utilizing a time-adapting threshold based on measurement data. The healthcare response includes one of a group selected from diagnostic testing and early goal-directed therapy in which sepsis-bundles are delivered.

In accordance with another aspect of the present invention, a system for predicting septic shock in a patient includes a display and a graphical user-interface. A non-transitory computer readable medium is programmed for acquiring data for the patient, wherein the data comprises physiological time-series (PTS) data and electronic health record (EHR) data. A risk score for the patient is determined at a predetermined time interval using a generalized linear model (GLM). The risk score is treated as the observable output of a hidden Markov model (HMM), using the HMM to estimate a transition probability that a patient has transitioned from a clinical state of sepsis to a pre-shock state. The transition probability is compared to a fixed threshold and the patient's condition is classified as septic shock if the patient reaches the fixed threshold, wherein the time at which the patient reaches the fixed threshold is defined as t_{d}. A healthcare response is triggered, if the patient reaches t_{d}.

In accordance with yet another aspect of the present invention, the non-transitory computer readable medium is programmed for triggering the display to show a septic shock warning alert that is positioned on top of any other information on the display. The non-transitory computer readable medium is programmed for requiring an authorized healthcare provider to certify that action has been taken before the septic shock warning alert can be moved. The PTS data includes heart rate, systolic blood pressure, partial pressure of oxygen in arterial blood, respiratory rate, Glasgow Coma Score, lactate level, blood urea nitrogen, white blood cell count, and respiratory, coagulatory and cardiovascular SOFA scores. The generalized linear model is defined as

$$\hat{P} ( t ) = \frac{{e}^{{β}_{0} + {\underline{β}}^{T} \underline{x} ( t )}}{1 + {e}^{{β}_{0} + {\underline{β}}^{T} \underline{x} ( t )}}$$

and the HMM is defined as π(t)=P(y(t)=1|x(t), x(t−1), . . . , x(1)). The PTS data is acquired at least every minute, and the risk score is calculated at least every minute. The risk score and transition probability are updated whenever a new clinical measurement becomes available in the PTS data or the EHR data. The transition probability is chosen to correspond to a point on a receiver operating curve (ROC) closest to a true positive rate (TPR)=0 and false positive rate (FPR)=0. Alternately, the transition probability is chosen based on a detection rule utilizing a time-adapting threshold based on measurement data.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIGS. 1A and 1B illustrate graphical views of the time-evolving risk score and transition probability for a patient with sepsis who does transition to septic shock (during the time interval shaded), and a patient with sepsis who does not transition to septic shock, respectively.

FIG. 2 illustrates a sample set of model coefficients for ten features identified by the algorithm of the present invention as yielding the greatest detection performance, in descending order of relative importance.

FIG. 3 illustrates a graphical view of ROC curves for detection methods with a risk score computed using either the method of the present invention or a Cox hazard model.

FIG. 4 illustrates a graphical view of a histogram of early warning times (EWTs).

FIGS. 5A-5D illustrate graphical views of a comparison of Sepsis-2 and Sepsis-3 clinical state label characteristics calculated from EHR and PTS data in the study population. FIG. 5A illustrates a time evolution of Sepsis-2 labels for subject 3205. FIG. 5B illustrates a Sepsis-2 state dwell time distributions for non-sepsis, sepsis/severe sepsis, and septic shock. Due to frequent fluctuations between sepsis/severe sepsis and non-sepsis in Sepsis-2, the relatively small number of occurrences of septic shock are not visible. FIG. 5C illustrates a time evolution of Sepsis-3 labels for subject 3205 FIG. 5D illustrates a Sepsis-3 state dwell time distributions for non-sepsis, sepsis, and septic shock.

FIGS. 6A and 6B illustrate graphical views of performance vs minimum dataset length. For each value of minimum dataset length, all datasets shorter than the minimum dataset length were excluded from the analysis. Mean values across all bootstrap iterations are indicated by the bold line, and 95% confidence intervals are indicated by the shaded area.

FIG. 7 illustrates a graphical view of merging electronic health record (EHR) data (indicated in the darker grey) and PTS data (indicated in the lighter grey) is accomplished by taking values from the PTS data wherever available, and from the EHR data where PTS data is not.

FIGS. 8A and 8B illustrate schematic diagrams of prediction method detailing the two steps involved in predicting impending transition to septic shock using physiological observations from PTS and EHR data x(t). FIG. 8A illustrates computation of the risk score z(t) using a generalized linear model that operates on input data x(t) consisting of features derived from patient EHR and PTS data. FIG. 8B illustrates the hidden Markov model (HMM) governing transition from the clinical state of sepsis (clinical state y(t)=0) to septic shock (clinical state y(t)=1).

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

The present invention is directed to a system and method for using physiological time-series (PTS) data sampled continuously from patients. An algorithm according to an embodiment of the present invention applies statistical modeling and machine learning methods to implement an early warning policy for predicting those patients likely to transition from non-sepsis, early sepsis or sepsis into septic shock. Results demonstrate that the system and method of the present invention can provide higher sensitivity and specificity in this task than any other method reported to date. It provides advanced warning of this pending transition with a median value being 12.5 hours, giving ample opportunity for physicians to intervene to prevent the patient from developing septic shock. This early warning time (EWT) is more than double that achieved using prior published methods (Cox model, median EWT 5.5 hours). A method according to the present invention includes the use of high frequency PTS data acquired at high rate (in this study every minute) from patients to do automated advanced warning of pending transitions in patient clinical state. A substantial window of early intervention is opened during which patients can be treated to reduce the likelihood of their transition to septic shock.

The foundation of the approach of the present invention is the assumption that there exists a clinical state of sepsis referred to as the “pre-shock” state. The existence of this pre-shock state is predicated on the fact that the physiology of sepsis patients who progress to septic shock must be gradually changing with time as their condition worsens, and therefore these patients will first transition into the pre-shock state before entering septic shock at time t_{d}. Note that on the basis of current accepted definitions of sepsis and septic shock, patients who enter what we call the pre-shock state are still diagnosed as having sepsis. They are however those patients with sepsis who are highly likely to transition at some future time point to septic shock. Early prediction of those patients who will ultimately develop septic shock therefore corresponds to identifying those patients who enter the pre-shock state. The time interval between the time at which a patient is clinically diagnosed as having septic shock and the time of entry into the pre-shock state is referred to as the early warning time (EWT). A hidden Markov model (HMM) that operates on a risk score calculated each minute from a set of features measured from patients is used to estimate the probability that a patient has transitioned from the state of sepsis to the sepsis pre-shock. The time of entry into the pre-shock state is defined as the time at which the transition probability exceeds a threshold value. This novel paradigm yields improved performance in early prediction of impending septic shock relative to existing methods, including more than a doubling of EWT.

In early 2016, an international task force of experts published a new consensus definition of sepsis known as Sepsis-3. Consensus definitions describe how a patient's clinical state (e.g., non-sepsis, sepsis, septic shock) can be labeled based on clinically measured variables. By applying the Sepsis-3 consensus definitions to appropriate, time-stamped clinical measurements, it is determined for this hypothetical patient that during time interval t=[0, t_{O}) the patient's clinical state is sepsis, and that at time to the patient transitions from the clinical state of sepsis to septic shock. In this general way, consensus definitions of clinical states can be applied to time-stamped clinical variables to label the clinical state of patients as a function of time. While consensus definitions can be used in conjunction with the present invention, it is also possible that any event or clinical label can also be used, as is known to or conceivable to one of skill in the art.

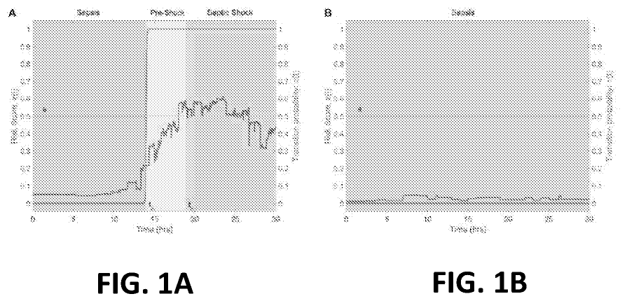

One key assumption of the framework of the present invention is that at some time during the interval [0, t_{O}) when the patient is clinically diagnosed as being in the state of sepsis (leftmost-shaded region, labeled “Sepsis”, FIG. 1A), the physiology of the patient begins to change as they transition towards the clinical state of septic shock (rightmost shaded region, labeled “Septic Shock”, FIG. 1A). In general, the hypothesis is that in those patients who transition from clinical state of sepsis to the clinical state of septic shock, there is some time t_{d} such that the statistical distribution of physiological measurements made during the interval [t_{d}, t_{O}) differ significantly from those during the interval [0, t_{d}), reflecting changes in the underlying physiology of the patient as the disease of sepsis evolves and their condition deteriorates. The time interval from [t_{d}, t_{O}) is defined as a new clinical state of sepsis referred to as the pre-shock state (middle region, labeled “Pre-Shock”, FIG. 1A). Patients only enter this state if at some future time they will transition from sepsis to the state of septic shock. Therefore, the time at which they enter the pre-shock state to is the time at which patients are identified as being at high risk for septic shock. The time interval tₒ−t_{d} is defined as the early warning time (EWT). The larger is EWT, the longer is the time-window of intervention to treat the patient to prevent their transition into a more serious clinical state.

Another key aspect of the framework of the present invention is that the transition into the pre-shock state is modeled using a hidden Markov model (HMM), where the observed variable is a time-evolving risk score z(t) generated by applying a logistic generalized linear model (GLM) to a set of features calculated each minute from patient PTS and EHR data. Optimal GLM weights are calculated from training data over a time window immediately preceding onset of septic shock. Using the HMM in which the observed variable is the GLM-based risk score, a Bayesian estimate of the transition probability π(t) can be calculated. The transition probability is a data-driven estimate of the probability that the patient has transitioned from the state of sepsis to the pre-shock state. The first time (the detection time t_{d}) at which this transition probability exceeds a fixed threshold defines the transition into the pre-shock state. FIGS. 1A and 1B are graphical views of exemplary risk score trajectories and transition probabilities from a patient who does (FIG. 1A) and one who does not (FIG. 1B) progress from sepsis to septic shock. FIGS. 1A and 1B also illustrate that there is a continuum between sepsis and septic shock. A patient can have sepsis without it developing into pre-shock or septic shock. Ideally, the present invention allows healthcare providers to intervene and treat patients with sepsis before it develops into septic shock.

The risk score is computed using continuously sampled physiological measurements from patients referred to as physiological time-series (PTS) data, and more slowly evolving variables extracted from that patients electronic health record (EHR). This risk score is updated every minute since that is the rate at which PTS data are acquired in this work. This risk score could be computed in many ways. One such way to compute the risk score is using a Generalized Linear Model (GLM) of the following form:

$$z ( x ( t ) ) = \frac{{e}^{{β}_{0} + {β}^{T} x ( t )}}{1 + {e}^{{β}_{0} + {β}^{T} x ( t )}}$$

where z(x(t)) is the risk score, β₀ is a constant, β is a k×1 vector of coefficients, “T” is the transpose operator, and x(t) is a k×1 vector of measured physiological variables as well as variables extracted from the electronic health record (EHR). Note that x(t) can be a derived function of the afore-mentioned variables, including functions of past values and variables reflecting treatment. These k variables are referred to as features. The k features include physiological time-series data measured from the patient at one-minute intervals, as well as variables from the EHR that are typically updated at much longer intervals. This enables the risk score to be updated at one-minute intervals. Because the risk score of the present invention is based on physiological variables measured very frequently, this increase the possibility for early detection of a clinical state change.

The GLM assumes that the clinical state labels at each time step (minute) are generated by independent samples of a Bernoulli random variable parameterized by the risk. That is, the clinical state labels over the window of interest for patients in sepsis who eventually transition to the pre-shock state are all denoted as 1, while the clinical state labels over the entire time window for patients who do not are all denoted as 0. In some embodiments, the clinical state can be defined by the user. The parameters {β₀, β₁, . . . } are estimated by maximizing the data likelihood function of observing the clinical state labels using a training patient cohort. The GLM is built using data from patients with sepsis who do and do not develop septic shock. Data from each patient over a selected time-window is used to build the GLM. For patients who transition to septic shock, this time-window begins prior to septic shock onset and ends just before septic shock onset. This avoids analyzing data from septic shock patients after they have been clinically labeled as being in septic shock, because part of the Sepsis-3 definition of septic shock is based on the actual treatment of these patients for septic shock. Data from time intervals following transition to septic shock therefore come from patients who are being treated for septic shock, and not from patients with septic shock who are not being treated for it.

At times tₙ the k features are observed from each patient and the risk is z(tₙ) computed as described above. This risk is assumed to be the observed output of a hidden Markov model (HMM) describing transition between the state of sepsis and the pre-shock state. These transition cannot be observed directly, they can only be inferred indirectly from the observed output z(t).

Using the HMM, a Bayesian estimate of each patient's probability of transition into the pre-shock state can be computed at each minute. Specifically, let y(t)=1 if the patient is in the pre-shock state, and let y(t)=0 if they are in the clinical state of sepsis. Define the transition probability π(t) as π(t)=P(y(t)=1|x(t), x(t−1), . . . , x(1)) be the probability that the patient has entered the pre-shock state by time t, conditioned on all past observations. Because each patient begins in the sepsis state, π(0)=0. A recursive formula for π(t) can then be derived as a function of t. One simple derivation is:

$$π ( t + 1 ) = \frac{\frac{q ( x ( t + 1 ) \vert y ( t + 1 ) = 1 )}{q ( x ( t + 1 ) \vert y ( t + 1 ) = 0 )} ( π ( t ) + ( 1 - π ( t ) ) p )}{( 1 - p ) ( 1 - π ( t ) ) + \frac{q ( x ( t + 1 ) \vert y ( t + 1 ) = 1 )}{q ( x ( t + 1 ) \vert y ( t + 1 ) = 0 )} ( π ( t ) + ( 1 - π ( t ) ) p )}$$

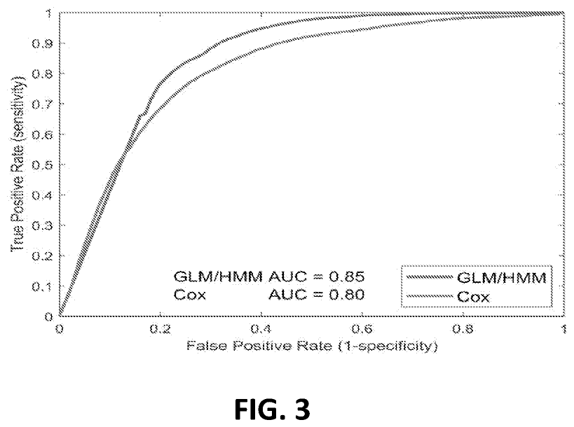

Detection occurs at the first time at which a patient's transition probability exceeds the threshold value, i.e. π(t)>θ, for a fixed-threshold θ. The time of threshold crossing is defined as the detection time, t_{d}. The optimal detection threshold is determined from the ROC curve illustrated in FIG. 3, as the value of the threshold corresponding to the point on the ROC curve closest to the upper left-hand corner. FIG. 3 illustrates a graphical view of ROC curves for detection methods with a risk score computed using either the method of the present invention or a Cox hazard model. Other definitions of the threshold can be defined by the user, these alternatives involve selecting other points on the ROC curve.

If the fixed threshold θ is reached, a healthcare response is triggered. This response can be triggered in any way known to or conceivable to one of skill in the art. In some instances, it is possible that a display is triggered to show a septic shock warning on top of any other data or images on the display. It is also possible that the warning cannot be displaced until an authorized healthcare provider notes that an appropriate action has been taken, via input to the system.

A number of computational approaches to early detection of sepsis and septic shock that leverage data from Electronic Health Records (EHRs) have been developed. In particular, one approach specifically targeted septic shock, using EHR data to identify patients with high risk of developing septic shock well before its onset. While these tools are successful in that they are able to identify at-risk patients to some extent, the EHR data upon which these tools rely is limited by the low frequency of data entries. Due to the rapid temporal evolution of septic shock, effective early detection cannot be based on EHR data that are updated infrequently, or on bioassays that take hours to perform or are too expensive to perform repeatedly at the necessary time scale. Intensive care unit (ICU) patients are heavily instrumented with a variety of sensors monitoring physiological functions. Physiological time-series (PTS) data generated by sampling these sensor signals at intervals ranging from milliseconds to minutes provide the highest-temporal-resolution view of a patient's state that can be achieved. A system that can leverage this information-rich data source in conjunction with the data available in the EHR will perform better than a method which relies on EHR data alone. Leveraging these data is a unique aspect of our approach. To test this hypothesis, a generalized linear model (GLM) is used to calculate a minute-by-minute risk score based on a combination of slowly-evolving EHR data as well as PTS data sampled at intervals of one minute. The risk model is applied to patient data, and a fixed-threshold decision rule is used to classify those patients with sepsis who are and are not likely to progress to septic shock. Results show that the resulting classifier has significantly higher sensitivity and specificity than do risk models based on EHR data alone. However, on average, the advanced warning of pending septic shock when using PTS and EHR data versus EHR data alone are similar.

A key assumption of the approach of the present invention is that in patients who transition from sepsis to septic shock, the clinical state of sepsis can be decomposed into two temporally adjacent sub-states. In FIGS. 1A and 1B, risk score over time is denoted by the variable medium grey-line, transition probability by the dark grey line, and threshold by the light grey horizontal line. Patient state transitions into the pre-shock state and the state of septic shock occur at times t_{d} and tₒ, respectively. Time is given in hours relative to the start of observations. FIG. 1A shows an example of a patient with sepsis who transitions to septic shock at time tₒ. The clinical condition of this patient was determined every minute by applying the Sepsis-3 definitions of sepsis and septic shock to EHR and PTS data from this patient(l).

Detection of impending septic shock (that is, the patient transitions from the state of sepsis to the pre-shock state) is considered to be a true positive event if the patient subsequently transitions to septic shock, and if the detection event occurs at least tₖ hours prior to tₒ. The parameter tₖ is referred to as the minimum actionable detection time, and represents the minimum time over which a patient intervention can be achieved. In an exemplary implementation, upon advice from critical care physicians, the time tₖ was set to 0.5 hours. If no detection event occurs prior to tₒ, or if the detection event occurs less than tₖ hours prior to tₒ, then the model prediction is considered to be a false negative case. Similarly, a true negative case occurs when there is no detection of septic shock for a patient who never entered septic shock, and a false positive case occurs when a septic shock detection event occurs for a patient who never entered septic shock. Early warning time (EWT) is defined as tₒ−t_{d}, the duration of the interval between the detection event and septic shock onset. The larger the value of EWT, the more advanced warning there is of a pending transition to septic shock.

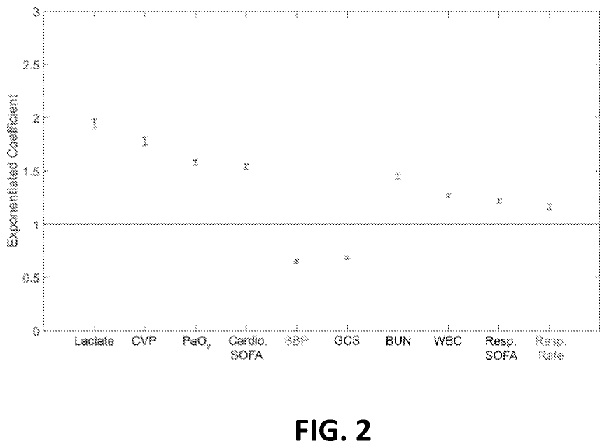

Of the 2926 patients included in testing of the present invention, using Sepsis-3 definitions 424 never entered sepsis, 2502 entered sepsis, and of these, 328 entered septic shock. Performance criteria are given as mean values computed from 100 iterations in which random 70:30 training-testing samples are drawn (i.e. for each iteration, 70% of the data is used for training, 30% is used for testing), where the model coefficients and detection threshold are learned from the training set, and performance criteria evaluated on the testing set. FIG. 2 illustrates a graphical view of exponentiated model coefficients and 95% confidence bounds for the 10 selected normalized features from one sample train/test iteration. These coefficients were learned using features normalized to have a mean of 0 and unit standard deviation. Candidate feature sets were pruned using lasso regularization. Coefficients are shown in descending order of importance from left to right. Based on the relative magnitude of the GLM-weights for each (normalized) feature, elevated lactate, a low-Glasgow Coma Score (GCS), and elevated cardiovascular Sequential Organ Failure Assessment (SOFA) score, and partial pressure of oxygen in blood (PaO₂) are the four most important indicators that a sepsis patient is at risk of entering septic shock.

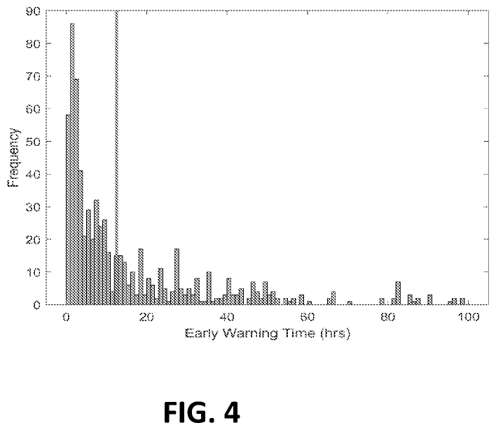

Using this method, septic shock can be detected with an area under the receiver operating characteristic (ROC) curve (area under curve, AUC) of 0.85, a sensitivity of 82%, and a specificity of 77%, as illustrated in FIG. 3. Clinical state labels were determined using Sepsis-3 criteria, and performance was evaluated using either the HMM/GLM method or Cox method used previously. Greatest AUC is achieved using an HMM/GLM (light grey). In FIG. 3, the true positive rate (TPR) is plotted against the false positive rate (FPR). FIG. 4 illustrates a graphical view of a histogram of EWTs. The dashed vertical line shows median value of 12.5. FIG. 4 shows the distribution of EWTs. The median EWT across all true positive cases is 12.5 hours (vertical dashed line; Interquartile range (IQR) 3.0 hours-55.0 hours). The Cox proportional hazards model for early detection of septic shock yielded a median EWT of 5.5 hours. The HMM/GLM method more than doubled EWT with 95% confidence.

The changing nature of patient features during the pre-shock state is shown in Table 1. The pre-shock state is physiologically distinct from both the sepsis state and the state of septic shock itself. Table 1 shows that the average values of the top six features from FIG. 2 (lactate, CVP, PaO₂, Cardiovascular SOFA score, Systolic Blood Pressure (SBP), Glasgow Coma Score (GCS)) exhibit statistically significant (α<0.01, Bonferroni corrected) increases upon transition from sepsis to the pre-shock state in a group of patients who all progress from sepsis to septic shock. Similarly, in accordance with the negative sign of their GLM coefficients, SBP and GCS show statistically significant decreases upon this transition. Similar changes indicative of a continuing trend in these top six features are observed upon transition from pre-shock to septic shock, with the exception of PaO₂, which increases in the interval preceding the pre-shock state, then decreases with septic shock onset.

TABLE 1
Physiological characterization of the pre-shock state.
Evolution of patient physiology during progression from sepsis to septic
shock for top six physiological features. Values are given as mean ±
standard deviation. Sixty data points were sampled from each of three
different time intervals in the same set of 61 patients (N = 3660 for
each clinical state), all of whom progress from sepsis to septic shock
during data acquisition and have a minimum of twelve hours of data
available prior to t_{d}. Sepsis data were sampled from the earliest hour
of observations available. Pre-shock data are sampled from the 1-
hour time interval immediately following detection time t_{d}. Septic
shock data are sampled uniformly from the time interval following
septic shock onset tₒ.

| Physiological | | | |
|---|---|---|---|
| Feature | Sepsis | Pre-shock | Septic shock |
| Lactate (mmol/L) | 2.32 ± 1.47 | 3.49 ± 2.77 | 4.39 ± 3.23 |
| CVP (mmHg) | 11.9 ± 5.8 | 13.7 ± 6.3 | 15.5 ± 6.0 |
| PaO₂ (mmHg) | 157.0 ± 10.4 | 183.8 ± 121.5 | 116.8 ± 55.5 |
| Cardio SOFA | 0.6 ± 1.2 | 1.3 ± 1.5 | 2.7 ± 1.3 |
| SBP (mmHg) | 80.5 ± 32.2 | 78.1 ± 27.7 | 72.9 ± 13.6 |
| GCS | 12.5 ± 3.8 | 9.2 ± 4.8 | 7.2 ± 3.9 |

FIG. 2 shows exponentiated model coefficients and 95% confidence bounds for the ten selected features from one sample train/test iteration. These coefficients were learned using features normalized to a mean of 0 and unit standard deviation. Features which are available in PTS data are labeled in red. Abbreviations: CVP—Central Venous Pressure; PaO2: Partial pressure of oxygen; Cardio SOFA—Cardiovascular SOFA Score; SBP—Systolic Blood Pressure; GCS—Glasgow Coma Scale; BUN—Blood Urea Nitrogen; WBC—White Blood Cell Count; Resp. SOFA—Respiratory SOFA Score; Resp. Rate—Respiratory Rate

The detection threshold applied to the HMM transition probability was chosen to correspond to the point on the ROC curve closest to the upper left-hand corner of the plot (i.e. where TPR=1 and FPR=0). It's possible that in practical usage, a different method of choosing the detection threshold by selecting a different point on the ROC curve may be preferred that balances the trade-off between sensitivity and specificity in a different way. In addition, a more sophisticated detection rule utilizing a time-adapting threshold based on measurement data may yield improved detection performance. In particular, the threshold may decrease over time if a high-risk patient remains in the sepsis state for a long period. Time-varying threshold policies such as those derived from quickest detection algorithms used to detect seizure events in epilepsy patients could also be leveraged.

The patient data sets used in this study are from the MIMIC-II database of adult ICU patients. These patients were admitted to ICUs having many different conditions. No attempt was made to stratify patients based on co-morbidities, and to develop optimal GLM weights β for each broad category of co-morbidity. This would have resulted in smaller training sets. With adequate data set size, such an approach would likely yield even better performance. Even though co-morbidities were not considered, the method described here achieves EWTs that are, for the most part, well before septic shock onset, with a median EWT of 12.5 hours. This provides ample time for intervention on the part of caregivers. The specific intervention to be made is a decision for the physician, and could include additional diagnostic tests and/or early goal-directed therapy in which sepsis-bundles are delivered rapidly following diagnosis of septic shock. Such therapy is known to reduce mortality, treatment costs and hospital readmissions. This particular data set and implementation is presented herein as an example. This implementation of the present invention is not meant to be considered limiting. The present invention can be implemented on any form of patient data collected on any type of clinical criteria or condition known to or conceivable to one of skill in the art.

Clinical data from the MIMIC-II database was previously used in order to predict patients at risk of developing septic shock, for which they report an AUC of 0.83, 85% sensitivity, 67% specificity, and a median detection time of 28 hours (IQR, 10.6-94.2 hours). This method, which was named a Targeted real-time early warning score (TREWScore), consists of a Cox proportional hazards model trained on features extracted from the EHR and time-to-septic-shock-onset values which they compute using the Sepsis-2 (rather than Sepsis-3) criteria for septic shock, where sepsis is defined as the presence of infection and systemic inflammatory response syndrome (SIRS). The Sepsis-2 definitions yield clinical state labels that fluctuate at a high rate over time—a property referred to as temporal instability of clinical state labels. To support more direct comparison with Sepsis-3, the sepsis and severe sepsis states as defined by Sepsis-2 criteria were combined into an aggregate state.

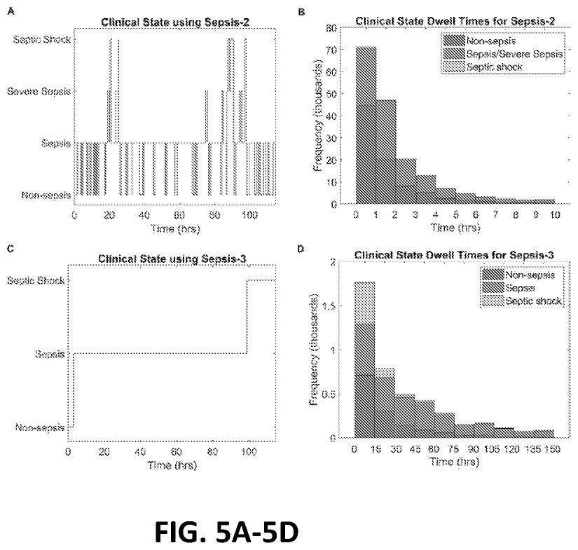

FIGS. 5A-5D illustrate graphical views of a comparison of Sepsis-2 and Sepsis-3 clinical state label characteristics calculated from EHR data in the study population. FIG. 5A illustrates a time evolution of Sepsis-2 labels for subject 3205. FIG. 5B illustrates a Sepsis-2 state dwell time distributions for non-sepsis, sepsis/severe sepsis, and septic shock. Due to frequent fluctuations between sepsis/severe sepsis and non-sepsis in Sepsis-2, the relatively small number of occurrences of septic shock are not visible. FIG. 5C illustrates a time evolution of Sepsis-3 labels for subject 3205 FIG. 5D illustrates a Sepsis-3 state dwell time distributions for non-sepsis, sepsis, and septic shock.

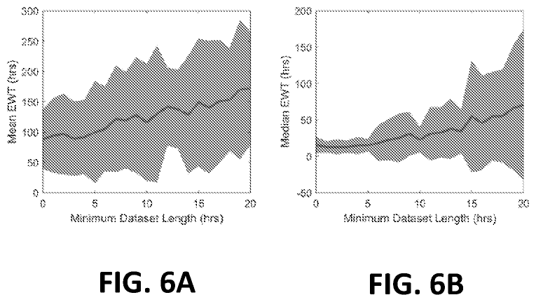

The mean number of label changes per patient in this same group of patients is 16.5, with a median of 8 when using Sepsis-2 criteria, whereas the mean number of label changes is 1.04 with a median of 0 when using Sepsis-3 criteria. The Sepsis-2-based clinical labels are temporally unstable, unlike those determined using the Sepsis-3 criteria. Clinical state labels change so frequently over time when using Sepsis-2 definitions that it is difficult to determine how the TREWScore study was done given it's impossible to know the true clinical state of the patients. Furthermore, when the Cox proportional hazards model decision approach employed in the TREWScore study is used, the median EWT was 5.5 hours, not the 28 hours reported in the TREWScore study. The duration of the available patient data preceding septic shock onset limits the maximum achievable EWT, as illustrated in FIGS. 6A and 6B. FIGS. 6A and 6B illustrate graphical views of performance vs minimum dataset length. For each value of minimum dataset length, all datasets shorter than the minimum dataset length were excluded from the analysis. Mean values across all bootstrap iterations are indicated by the bold line, and 95% confidence intervals are indicated by the shaded area. A median EWT of 28 hours is reported when using the SIRS-based Sepsis-2 criteria.

There was an attempt to reproduce this finding by generating clinical state labels using the same Sepsis-2 clinical criteria employed in Henry et al. rather than the Sepsis-3 criteria used in this study. However, the temporal instability (see FIGS. 5A-5D) of Sepsis-2 clinical labels makes it difficult to reliably identify when a patient is in septic shock. The time interval between the first measured data point and time of septic shock onset (referred to as “dataset length”) is an upper bound on EWT. Median dataset length also sets the upper bound on median EWT. When Sepsis-3 diagnostic criteria are used, median dataset length and thus the maximum possible median EWT is 23.6 hours. To further illustrate the effect of dataset length on EWT (FIGS. 6A and 6B), analyses were repeated while excluding datasets shorter than a given minimum length. As minimum dataset length increases, median EWT increases from 12.5 hours to 50 hours as shorter datasets are excluded. In addition, ˜30% of the true positive detections occur in the first minute of patient observations, indicating that patients have already entered the pre-shock state at the time of ICU admission. In these cases, had data been available from earlier times, the EWT achieved would have been greater. These findings point out that continuous collection and analysis of patient EHR and PTS data is necessary to achieve the maximum EWT.

The present invention presents a novel approach to the prediction of those patients with sepsis who are likely to transition to septic shock. The key hypothesis underlying the approach of the present invention is that in those patients who transition from sepsis to septic shock, the sepsis state can be sub-divided into temporally-adjacent clinical states of sepsis followed by a state called the pre-shock state. Intuitively, the pre-shock state corresponds to a time interval during which the patients' condition is worsening, however they still have not transitioned into septic shock. In this formulation, the early detection paradigm corresponds to estimating the time at which the patient enters this pre-shock state. Results presented here show that this can be done by computing a risk-score using a generalized linear model, treating that risk as the observable output of a hidden Markov model, using the HMM model to estimate the probability that a patient has transitioned from the clinical state of sepsis to the pre-shock state (the transition probability), and comparing the transition probability to a fixed threshold. Performance achieved has relatively high sensitivity and specificity, and the median early warning is 12.5 hours, providing adequate time to intervene and treat the patient before they enter septic shock. The median early warning can be as large as 50-hours when only sufficiently long data sets are considered. This paradigm is general and can be applied to many other patient clinical state transition detection problems in critical care units.

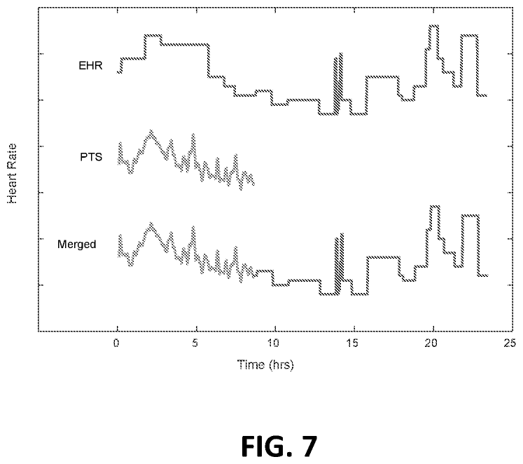

As shown in FIG. 7, a clinical variable (e.g. HR) is occasionally available over a limited time window as minute-to-minute PTS data, but outside that time window, is only available as occasional entries in the EHR. FIG. 7 illustrates a graphical view of merging EHR data (indicated in the darker grey) and PTS data (indicated in the lighter grey) is accomplished by taking values from the PTS data wherever available, and from the EHR data where PTS data is not. When this happens, EHR and PTS data are merged by using values from the PTS data wherever available, and using values of the resampled EHR data elsewhere. For the comparison studies where EHR data only were used, this last step of merging PTS and EHR data was omitted.

In order to determine sepsis and septic-shock onset times, the Sepsis-3 criteria were applied to the EHR data extracted from the MIMIC-II database. A patient is considered to be in sepsis if they have suspected infection, as determined by their ICD-9 codes, and a sequential organ failure assessment (SOFA) score of 2 or higher. SOFA score is evaluated each time a new clinical measurement involved in calculating the score is available. This calculation is done using the worst observed value of that measurement over the past 24 hours. A patient is considered to be in septic shock if they fulfill all of the following criteria: they have sepsis; have been adequately fluid resuscitated; and require vasopressors to maintain a mean arterial blood pressure of at least 65 mm-Hg; and have a serum lactate >2 mmol/L. The vasopressors considered are dopamine, dobutamine, epinephrine, norepinephrine, and phenylephrine. The definition of adequate fluid resuscitation comes from the 2016 Surviving Sepsis Campaign guidelines for treatment, which recommend 30mL/kg of fluids over three hours, and have treatment targets of urine output >0.5mL/kg/hr and CVP of 8-12 mmHg. Based on this definition, a patient is considered adequately fluid resuscitated if, in the past three hours, they have been administered at least 30 mL/kg of fluids, or if the treatment targets of urine output >0.5 mL/kg/hr or CVP 8-12 mmHg have been met. The time of septic shock onset is then determined as the first time at which a patient was determined to be in septic shock. Of the 2,926 patients with suspected infection, septic shock was determined in 328, sepsis in 2,174, and no sepsis in 424 (See Table 3 for additional demographic information).

To calculate the risk score separating shock patients from non-shock patients, a generalized linear model (GLM) for Bernoulli observations of patient features is applied. pᵢ(t) is defined as the probability that patient i is in the sepsis sub-state T at time t, conditioned on being in the clinical state of sepsis. Specifically, at a given minute, each patient's classification is a Bernoulli random variable denoted by yᵢ(t)∈[0,1} where yᵢ(t)=1 means that at time t, patient i is in the pre-shock state, and thus, highly likely to enter septic shock, and yᵢ(t)=0 means that at time t, patient i is in the sepsis state. pᵢ(t) is then described as a function of xᵢ(t), pᵢ(t)=g(xᵢ(t)), where xᵢ(t) is the vector of time-evolving features derived from PTS and EHR data that influence Pr(yᵢ=1|sepsis)≙pᵢ. It is important to note that yᵢ and xᵢ change over time at the frequency with which they are measured, here the highest rate of measurement is per minute. The GLM framework ensures that a class of functions that are bounded between 0 and 1 and that render a concave likelihood function (has a unique global maximum) that can be efficiently maximized over an unknown set of parameters in the vector β.

In particular, the GLM is specified as follows:

$$Pr ( {y}_{i} ( t ) = 1 ) {=}^{Δ} {p}_{i} {=}^{Δ} g ( {x}_{i} ( t ) , β ) = \frac{{e}^{{\underline{β}}^{T} \underline{{x}_{i}} ( t )}}{1 + {e}^{{\underline{β}}^{T} \underline{{x}_{i}} ( t )}}$$

Moreover, a GLM has the advantages of allowing for fast computation of β as the maximum likelihood estimator (MLE), and for yielding a risk score that is easily interpretable in the clinical context. For instance, if all variables have been normalized to a mean of 0, and a standard deviation of 1, the magnitude and sign of the model coefficient in β corresponding to a given feature indicates its relative contribution to the risk of a patient being in sepsis sub-state T, and thus of entering septic shock. The larger the magnitude, the larger its relative contribution. A positive coefficient for a given feature means that when that feature is large, the risk of being in the pre-shock state is higher, and a negative coefficient means that when that feature is high, the risk of being in the pre-shock state is lower.

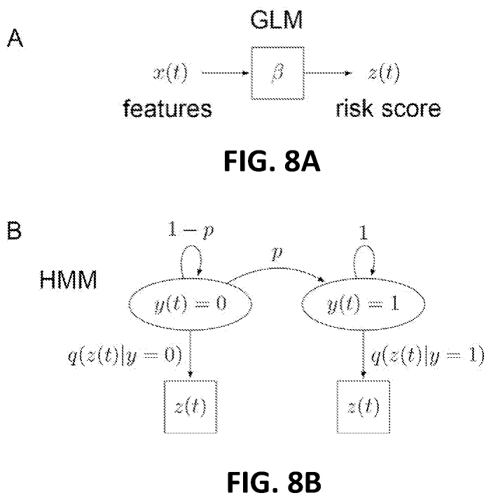

In patients who transition from sepsis to septic shock, there exists a clinical state of sepsis that is referred to as the “pre-shock” state. A hidden Markov model of this state transition is defined, where the observed variable is a GLM-based risk score, as illustrated in FIGS. 8A and 8B. π(t), the probability that the patient has transitioned into the pre-shock state is then estimated based on the observations of z(t), the risk score which is in turn calculated from PTS and EHR data. FIGS. 8A and 8B illustrate schematic diagrams of prediction method detailing the two steps involved in predicting impending transition to septic shock using physiological observations from PTS and EHR data x(t). From these physiological observations, a GLM is used to compute a univariate risk score z(t), as illustrated in FIG. 8A. This risk score z(t) is then defined as the observed variable for an HMM with two hidden states (y(t)=0 representing the state of sepsis and y(t)=1 representing the pre-shock state), as illustrated in FIG. 8B. The distribution of z(t) depends only on the state of the patient, and its conditional probability density function is given by q(z(t)|y(t)).

Estimation of the parameters of the HMM is done via maximum likelihood estimation. Let n₀ be the number of training data points such that yᵢ=0, and n₁ be the number of training data points such that yᵢ=1:

$${\hat{μ}}_{0} = \frac{1}{{n}_{0}} {\sum }_{{y}_{i} = 0}^{1} z ( {x}_{i} ) , {\hat{μ}}_{1} = \frac{1}{{n}_{1}} {\sum }_{{y}_{i} = 1} z ( {x}_{i} )$$

$${\hat{σ}}_{0}^{2} = \frac{1}{{n}_{0}} {\sum }_{{y}_{i} = 0} {( z ( {x}_{i} ) - {\hat{μ}}_{0} )}^{2} , {\hat{σ}}_{1}^{2} = \frac{1}{{n}_{1}} {\sum }_{{y}_{i} = 1} {( z ( {x}_{i} ) - {\hat{μ}}_{1} )}^{2}$$

The prior probability of state transition p is estimated as 1 /μ_{T}, where μ_{T} is the average length of observations, in minutes, before septic shock onset. This fully characterizes the HMM:

$$q ( z ( t ) \vert y ( t ) = 0 ) = \frac{1}{\sqrt{2 π {\hat{σ}}_{0}^{2}}} {e}^{\frac{- {( z ( t ) - {\hat{μ}}_{0} )}^{2}}{2 {\hat{σ}}_{0}^{2}}} , \\ q ( z ( t ) \vert y ( t ) = 1 ) = \frac{1}{\sqrt{2 π {\hat{σ}}_{1}^{2}}} {e}^{\frac{- {( z ( t ) - {\hat{μ}}_{1} )}^{2}}{2 {\hat{σ}}_{1}^{2}}}$$

For early prediction of septic shock, each patient's risk score is calculated for each minute of data from the beginning of their observations until septic shock onset. Using the HMM, a Bayesian estimate of each patient's probability of transition into the pre-shock state can be computed at each minute.

Specifically, let the transition probability π(t)=P(y(t)=1|x(t), x(t−1), . . . , x(1)) be the probability that the patient has entered the pre-shock state by time t, conditioned on all past observations. Because each patient begins in the sepsis state, π(0)=0. A recursive formula for π(t) can then be given for all subsequent values of t. Several derivations of this formula are possible One simple derivation is:

$$π ( t + 1 ) = \frac{\frac{q ( x ( t + 1 ) \vert y ( t + 1 ) = 1 )}{q ( x ( t + 1 ) \vert y ( t + 1 ) = 0 )} ( π ( t ) + ( 1 - π ( t ) ) p )}{( 1 - p ) ( 1 - π ( t ) ) + \frac{q ( x ( t + 1 ) \vert y ( t + 1 ) = 1 )}{q ( x ( t + 1 ) \vert y ( t + 1 ) = 0 )} ( π ( t ) + ( 1 - π ( t ) ) p )}$$

Detection occurs at the first time at which a patient's transition probability exceeds the threshold value, i.e. π(t)>θ, for a fixed threshold θ. This time of threshold crossing is defined as the detection time t_{d}. The optimal detection threshold is determined from the ROC curve as the value of the threshold corresponding to the point on the ROC curve closest to the upper left-hand corner. Early warning time (EWT) is defined as the difference between onset time to and detection time t_{d}.

In the implementation of TREWScore used herein, the same feature vector xᵢ(t) is used for learning a Cox proportional hazards model. In TREWScore, the risk of a patient developing septic shock conditioned on observations of their clinical features at a given time, denoted by λ(t|xᵢ(t)), is modeled as follows:

$$λ ( t \vert \underline{{x}_{i}} ( t ) ) = {λ}_{0} ( t ) {e}^{{\underline{β}}^{T} \underline{{x}_{i}} ( t )}$$

Estimation of β, however, is not accomplished using the binary labels yᵢ(t)∈{0,1}, but rather using the time until onset of septic shock. These feature-time-to-onset pairs are used in order to estimate β.

The results herein are exemplary and not meant to be considered limiting. The results are based on 100 iterations of repeated 70:30 training-testing samples, where in each iteration, the dataset is split into two cohorts, the first containing 70% of patients in the dataset, and the second containing 30% of patients the dataset. Each iteration has this sample taken independently of the other iterations. For each iteration, all models and thresholds are learned from the first cohort containing 70% of the data, which are referred to as the training set. Performance criteria are then evaluated using these models and thresholds on the second cohort containing 30% of the data, which are referred to as the testing set.

The algorithm of the present invention is trained on sepsis data from patients who never go into septic shock against sepsis data from septic shock patients in the modeling window from 2 hours before septic shock onset until 1 hour before septic shock onset. Specifically, when estimating the model coefficients β via MLE, the clinical features xᵢ(t) is used from patients in the training set who never enter septic shock, where the clinical labels, as determined by Sepsis-3, indicate sepsis, and assign the label yᵢ(t)=0 to all data points taken from those patients. The values of the clinical features xᵢ(t) are then taken from patients in the training set who develop septic shock from the time window spanning tₒ−2 hours to t₀−1 hour, where the clinical labels indicate sepsis, and assign the label yᵢ(t)=1 to all data points taken from those patients.

The decision to train the model of the present invention using data from a window of time during the sepsis sub-state T stems from the insight that septic shock, per the Sepsis-3 definitions, is a treated state; patients who fulfill the Sepsis-3 criteria for septic shock have been administered vasopressors and fluids, and thus, physiological data from the septic shock clinical state would reflect a perturbed view of septic shock as a result of the treatment given(l). In an attempt to characterize an unperturbed state indicative of imminent septic shock, the time windows surrounding the time of septic shock onset were examined to find that physiological data obtained from the sepsis state immediately preceding septic shock onset in septic shock patients was separable using a GLM-based risk score determined using data from the sepsis state in patients who never entered septic shock. The window was chosen to be between tₒ−2 and tₒ−1 because, out of the 1-hour wide windows surrounding septic shock onset, this window yielded the greatest detection performance as measured by AUC (FIGS. 10A and 10B).

To ensure that the learned model is not biased towards patients with a longer set of observations, particularly in the set of patients who do not develop septic shock and yᵢ(t)=0, each non-septic-shock dataset is resampled by selecting a random set of 180 data points from the available observations for that patient. If fewer than 180 minutes of observations are available for a patient, then this sampling is done with replacement. Each data point from the septic shock patients is repeated in the modeling window three times, so that each septic shock patient has the same number of data points per patient in the training set as the non-shock patients. With this resampled set of feature-label pairs the model is able to learn the parameters for the GLM via MLE; i.e. given y consisting of all yᵢ(t) in the resampled training set, β is chosen to maximize the data likelihood function:

$$Pr ( {y}_{i} ( t ) = 1 ) = \frac{{e}^{{\underline{β}}^{T} \underline{{x}_{i}} ( t )}}{1 + {e}^{{\underline{β}}^{T} \underline{{x}_{i}} ( t )}}$$

$$Pr ( {y}_{i} ( t ) = 0 ) = \frac{1}{1 + {e}^{{\underline{β}}^{T} \underline{{x}_{i}} ( t )}}$$

Assuming that each yᵢ(t) is independent, Pr (y|β) is evaluated as the product of the individual likelihoods:

$$\underline{β} = \begin{matrix}argmax \\ β\end{matrix} \{ Pr ( \underline{y} \vert β ) \}$$

Where, as defined by the Bernoulli GLM

$$Pr ( \underline{y} \vert β ) = {\prod }_{i , t} Pr ( {y}_{i} ( t ) \vert β )$$

For the implementation of TREWScore, xᵢ(t) from patients in the training set who do not enter septic shock is used labels yᵢ(t)>t_{end}−t, where t_{end} denotes the time at which the last set of observations for a patient is made are assigned. These data are right-censored, which means that the time until septic shock onset is not definite, but merely lower bounded by the time until the end of observations, as it is known that septic shock did not occur within the observed window.

A set of over 40 variables from the EHR and available PTS data are queried, 10 features are selected from this set that best characterize Sₓ^{T}. This is accomplished via lasso regression for both the GLM and the Cox model. In each case, 10 features are chosen by increasing the weight of the regularization term until only 10 non-zero features remained.

Patients typically undergo many treatments upon entering the ICU that perturb their physiological state. Therefore, a delay is taken in computing the risk score of two and a half hours before making any predictions. This allows the physiological state of new ICU patients to stabilize. This decreases the number of false positives, and results in a ˜2-3% improvement in detection specificity. Furthermore, a minimum actionable detection time tₖ is chosen such that if a detection event occurs after tₒ−tₖ, the detection event is considered to be a false negative. The parameter tₖ represents the width of a time interval that is too narrow to allow for any meaningful intervention to be made. Septic shock patients with no observations preceding septic shock onset, or patients with less than 3 hours total of observations are excluded from analysis. Detection is impossible in the absence of observations of patient features; in the case of septic shock patients with no observations preceding septic shock onset, early detection of septic shock is inherently possible for this reason. In the latter case of patients with less than 3 hours of observations, ignoring the first 2.5 hours of measurements and using the minimum detection bound of 0.5 hours similarly results in no data for analysis, and thus, early detection of septic shock is not possible using the chosen modeling parameters in these cases.

EHR features were queried from the MIMIC-II PostgreSQL database. Multiple items may correspond to the same feature; for these features, all item ids specified in Table 2 were queried. In the case of the administration of medication, some items report dosages in varying units of measure. All values were converted to mcg/kg/min. Similarly, temperature was sometimes reported in degrees Celsius, and sometimes in degrees Fahrenheit. For these features, the unit of measure for a given item id was determined, and the values converted to degrees Fahrenheit (either would have sufficed; it only matters that the values are all on the same unit of measure).

TABLE 2

| | Chart Events | Med Events |
|---|---|---|
| Feature | item id | item id |
| Heart rate | 211 | |
| Respiratory rate | 618 | |
| Temperature | 676, 677, 678, 679 | |
| SBP/DBP* | 51, 6701, 6926, 455 | |
| Mean BP | 52, 6702, 6927, 456 | |
| CVP | 113 | |
| PaO₂ | 490, 779 | |
| FiO₂ | 190, 3420 | |
| GCS | 198 | |
| Bilirubin | 4948, 848 | |
| Platelets | 828 | |
| Creatinine | 791, 3750, 1525 | |
| Lactate | 1531, 818 | |
| BUN | 781, 1162, 5876, 3737 | |
| Arterial pH | 1126, 4753 | |
| WBC | 861, 1127, 1542, 4200 | |
| PaCO₂ | 778 | |
| Respiratory Support | 3605 | |
| Hemoglobin | 814 | |
| Hematocrit | 3761, 813 | |
| Potassium | 829 | |
| Epinephrine | | 44, 119, 309 |
| Dopamine | | 43, 307 |
| Dobutamine | | 42, 306 |
| Norepinephrine | | 47, 120 |
| Phenylephrine | | 127, 128 |

Table 2 lists item ids for patient features queried from the MIMIC-II clinical database. *SBP and DBP are given in the same item in the MIMIC-II chart events database table; the value of SBP is given in the value 1 column, and the value of DBP is given in the value 2 column.

Fluid administration and urine output were calculated from the io events database table. Age, weight, and gender were determined. Charlston comorbidity index was calculated from ICD-9 codes.

In addition to the one-hour wide modeling windows, using windows of variable width ending at to was also explored. There was little variation in detection performance as the width of this window varied. However, this is not necessarily because all of the data in the time preceding septic shock is equal in predictive value; rather, since different patients have varying amounts of data available, most of the data in the modeling window will be from immediately preceding septic shock. This essentially dilutes any change in predictive value caused by data from different time points, as all modeling windows contain mostly data from the time immediately preceding septic shock onset.

TABLE 3
Demographic information for the 2926 patients included in the study.

| Statistic | Cohort | |
|---|---|---|
| Gender | No sepsis | |
| | Sepsis | |
| | Septic Shock | |
| Age, mean (SD) | No sepsis | 63.6 (21.9) |
| | Sepsis | 65.5 (18.4) |
| | Septic Shock | 66.9 (18.0) |
| Length of ICU stay, median days | No sepsis | |
| | Sepsis | |
| | Septic Shock | |
| Charlston comorbidity index, | No sepsis | 3.87 (3.54) |
| mean (SD) | | |
| | Sepsis | 4.58 (3.74) |
| | Septic Shock | 3.89 (3.53) |

The present invention can also take the form of a system with a display and a graphical user interface. Septic shock warnings can be shown on the display and the graphical user interface can be used to confirm that action is being taken with respect to the septic shock warning. In some instances, the septic shock warning can appear on the screen on top of any other information being displayed by the screen. In other cases, the septic shock warning can be moved to the top of the display to share space with other vital information for the patient. In some embodiments, the septic shock warning cannot be moved from its position on the screen until an authorized healthcare provider verifies that action is being taken with respect to the septic shock warning. The system can also include sensors that are configured to collect data at a high rate of frequency. Any noise from these sensors is corrected by the system of the present invention, before the risk score is calculated. The system can also be configured to calibrate these sensors from time to time.

The processing and display function of the present invention can be carried out using a computing device and a non-transitory computer readable medium. A non-transitory computer readable medium is understood to mean any article of manufacture that can be read by a computer. Such non-transitory computer readable media includes, but is not limited to, magnetic media, such as a floppy disk, flexible disk, hard disk, reel-to-reel tape, cartridge tape, cassette tape or cards, optical media such as CD-ROM, writable compact disc, magneto-optical media in disc, tape or card form, and paper media, such as punched cards and paper tape. The computing device can take any form known to or conceivable to one of skill in the art, such as a smartphone, tablet, phablet, personal computer, laptop, server, or cellular telephone.

The computing device may be a general computing device, such as a personal computer (PC), a UNIX workstation, a server, a mainframe computer, a personal digital assistant (PDA), smartphone, cellular phone, a tablet computer, a slate computer, or some combination of these. Alternatively, the computing device may be a specialized computing device conceivable by one of skill in the art. The remaining components may include programming code, such as source code, object code or executable code, stored on a non-transitory computer readable medium that may be loaded into the memory and processed by the processor in order to perform the desired functions of the system. The user interface device, which will be described in more detail herein, can include a cellular telephone, a smart phone, a tablet computing device, a pager, a PC computing device, laptop, or any other suitable device known to or conceivable by one of skill in the art.

A user interface device and the computing device may communicate with each other over a communication network via their respective communication interfaces. The communication network can include any viable combination of devices and systems capable of linking computer-based systems, such as the Internet; an intranet or extranet; a local area network (LAN); a wide area network (WAN); a direct cable connection; a private network; a public network; an Ethernet-based system; a token ring; a value-added network; a telephony-based system, including, for example, T1 or E1 devices; an Asynchronous Transfer Mode (ATM) network; a wired system; a wireless system; an optical system; cellular system; satellite system; a combination of any number of distributed processing networks or systems or the like.

The computing device can include a processor, a memory, a communication device, a communication interface, an input device, and a communication bus, respectively. The processor, may be executed in different ways for different embodiments of the computing device. One option is that the processor, is a device that can read and process data such as a program instruction stored in the memory, or received from an external source. Such a processor, may be embodied by a microcontroller. On the other hand, the processor may be a collection of electrical circuitry components built to interpret certain electrical signals and perform certain tasks in response to those signals, or the processor may be an integrated circuit, a field programmable gate array (FPGA), a complex programmable logic device (CPLD), a programmable logic array (PLA), an application specific integrated circuit (ASIC), or a combination thereof. Different complexities in the programming may affect the choice of type or combination of the above to comprise the processor.

Similarly to the choice of the processor, the configuration of a software of the user interface device and the computing device (further discussed herein) may affect the choice of memory used in the user interface device and the computing device. Other factors may also affect the choice of memory, type, such as price, speed, durability, size, capacity, and re-programmability. Thus, the memory, of the computing device may be, for example, volatile, non-volatile, solid state, magnetic, optical, permanent, removable, writable, rewriteable, or read-only memory. If the memory is removable, examples may include a CD, DVD, or USB flash memory which may be inserted into and removed from a CD and/or DVD reader/writer (not shown), or a USB port (not shown). The CD and/or DVD reader/writer, and the USB port may be integral or peripherally connected to user interface device and the computing device.

In various embodiments, user interface device and the computing device may be coupled to the communication network by way of the communication device. In various embodiments the communication device can incorporate any combination of devices—as well as any associated software or firmware—configured to couple processor-based systems, such as modems, network interface cards, serial buses, parallel buses, LAN or WAN interfaces, wireless or optical interfaces and the like, along with any associated transmission protocols, as may be desired or required by the design.

Working in conjunction with the communication device, the communication interface can provide the hardware for either a wired or wireless connection. For example, the communication interface, may include a connector or port for an OBD, Ethernet, serial, or parallel, or other physical connection. In other embodiments, the communication interface, may include an antenna for sending and receiving wireless signals for various protocols, such as, Bluetooth, Wi-Fi, ZigBee, cellular telephony, and other radio frequency (RF) protocols. The user interface device and the computing device can include one or more communication interfaces, designed for the same or different types of communication. Further, the communication interface, itself can be designed to handle more than one type of communication.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention. While exemplary embodiments are provided herein, these examples are not meant to be considered limiting. The examples are provided merely as a way to illustrate the present invention. Any suitable implementation of the present invention known to or conceivable by one of skill in the art could also be used.

1. A method for predicting septic shock in a patient comprising:
acquiring data for the patient, wherein the data comprises physiological time-series (PTS) data and electronic health record (EHR) data;
determining a risk score for the patient at a predetermined time interval using a generalized linear model (GLM);
treating the risk score as an observable output of a hidden Markov model (HMM), using the HMM to estimate a transition probability that a patient has transitioned from a clinical state of sepsis to a pre-shock state,
comparing the transition probability to a fixed threshold;
classifying the patient as one who will subsequently transition to septic shock if the patient reaches the fixed threshold, wherein the time at which the patient reaches the fixed threshold is defined as t_{d}; and,
triggering a healthcare response if the patient reaches t_{d}.

2. The method of claim 1 wherein the PTS data includes heart rate, systolic blood pressure, partial pressure of oxygen in arterial blood, respiratory rate, Glasgow Coma Score, lactate level, blood urea nitrogen, white blood cell count, and respiratory, coagulatory, and cardiovascular SOFA scores.

3. The method of claim 1 wherein the GLM comprises
$$\hat{P} ( t ) = \frac{{e}^{{β}_{0} + {\underline{β}}^{T} \underline{x} ( t )}}{1 + {e}^{{β}_{0} + {\underline{β}}^{T} \underline{x} ( t )}}$$
and the HMM comprises π(t)=P(y(t)=1|x(t), x(t−1), . . . , x(1)).

4. The method of claim 1 wherein the PTS data is acquired at a high rate, at least every minute.

5. The method of claim 4 wherein the risk score is calculated at least every minute.

6. The method of claim 4 wherein the PTS data is being updated continuously.

7. The method of claim 1 wherein the risk score is updated whenever a new clinical measurement becomes available in the PTS data or the EHR data.

8. The method of claim 1 wherein the transition probability is chosen to correspond to a point on a receiver operating curve (ROC) where true positive rate (TPR)=0 and false positive rate (FPR)=0.

9. The method of claim 1 wherein the transition probability is chosen based on a detection rule utilizing a time-adapting threshold based on measurement data.

10. The method of claim 1 wherein the healthcare response includes one of a group selected from diagnostic testing and early goal-directed therapy in which sepsis-bundles are delivered.

11. A system for predicting septic shock in a patient comprising:
a display;
a graphical user-interface;
a non-transitory computer readable medium programmed for:
acquiring data for the patient, wherein the data comprises physiological time-series (PTS) data and electronic health record (EHR) data;
determining a risk score for the patient at a predetermined time interval using a generalized linear model (GLM);
treating the risk score as an observable output of a hidden Markov model (HMM), using the HMM to estimate a transition probability that a patient has transitioned from a clinical state of sepsis to a pre-shock state,
comparing the transition probability to a fixed threshold;
classifying the patient as one who will subsequently transition to septic shock if the patient reaches the fixed threshold, wherein the time at which the patient reaches the fixed threshold is defined as t_{d}; and,
triggering a healthcare response if the patient reaches t_{d}.

12. The system of claim 11 further comprising the non-transitory computer readable medium being programmed for triggering the display to show a septic shock warning alert that is positioned on top of any other information on the display.

13. The system of claim 12, wherein the non-transitory computer readable medium is programmed for requiring an authorized healthcare provider to certify that action has been taken before the septic shock warning alert can be moved.

14. The system of claim 11 wherein the PTS data includes heart rate, systolic blood pressure, partial pressure of oxygen in arterial blood, respiratory rate, Glasgow Coma Score, lactate level, blood urea nitrogen, white blood cell count, and respiratory, coagulatory, and cardiovascular SOFA scores.

15. The system of claim 11 wherein the GLM comprises
$$\hat{P} ( t ) = \frac{{e}^{{β}_{0} + {\underline{β}}^{T} \underline{x} ( t )}}{1 + {e}^{{β}_{0} + {\underline{β}}^{T} \underline{x} ( t )}}$$
and the HMM comprises π(t)=P(y(t)=1|x(t), x(t−1), . . . , x(1)).

16. The system of claim 11 wherein the PTS data is acquired at least every minute.

17. The system of claim 11 wherein the risk score is calculated at least every minute.

18. The system of claim 11 wherein the risk score is updated whenever a new clinical measurement becomes available in the PTS data or the EHR data.

19. The system of claim 11 wherein the transition probability is chosen to correspond to a point on a receiver operating curve (ROC) where true positive rate (TPR)=1 and false positive rate (FPR)=0.

20. The system of claim 11 wherein the transition probability is chosen based on a detection rule utilizing a time-adapting threshold based on measurement data.
